(19) United States
(12) Patent Application Publication
(10) Pub. No.: US 2024/0082042 A1
(43) Pub. Date: Mar. 14, 2024

(54) NASAL DILATOR

(71) Applicant: Teruko SAKAGUCHI, Saitama-shi, Saitama (JP)

(72) Inventor: Teruko SAKAGUCHI, Saitama-shi, Saitama (JP)

(21) Appl. No.: 18/257,087

(22) PCT Filed: Jul. 26, 2021
(86) PCT No.: PCT/JP2021/027514

§ 371 Date: Jun. 12, 2023

(30) Foreign Application Priority Data
Dec. 14, 2020 (JP) 2020-206944

(51) Int. Cl. A61F 5/08

(52) U.S. Cl. CPC A61F 5/08; A61F 2210/0076

(57) ABSTRACT

This invention relates to a nasal dilator. An object of this invention is to provide a nasal dilator wherein adhesive substance layer is prevented from adhering to a top region of a nose bridge, and which does not need a dedicated member for preventing the adhesion of the substance layer to the top region of the nose bridge. The object is achieved by a nasal dilator comprising a flexible strip, an adhesive substance layer formed on one surface of the flexible strip and a release liner covering the adhesive substance layer, wherein the adhesive substance layer is formed on entire surface of said one surface of the flexible strip, the release liner is divided into three pieces, a first of which covers one longitudinal end region of the adhesive substance layer, a second of which covers longitudinal middle region of the adhesive substance layer and a third of which covers another longitudinal end region of the adhesive substance layer.

### TECHNICAL FIELD

This invention relates to a nasal dilator.

### BACKGROUND ART

Patent literature 1 discloses a nasal dilator comprising a flexible strip, an adhesive substance layer formed on one surface of the flexible strip and a release liner divided into two pieces and covering the adhesive substance layer. When the nasal dilator is used, both pieces of the release liner are removed from the adhesive substance layer, and then the adhesive substance layer is adhered to the nose bridge.

In the nasal dilator of Patent literature 1, a pad or a net is adhered to a longitudinal middle region of the adhesive substance layer so as to prevent adhesion of the adhesive substance layer to a top region of the nose bridge, thereby preventing peeling of skin of the top region of the nose bridge and damage of the top region of the nose bridge at the time of removal of the nasal dilator from the nose bridge.

### CITATION LIST

### Patent Literature

PTL 1: Japanese Patent Laid-Open No. 2005-087766

### SUMMARY OF INVENTION

### Technical Problem

The nasal dilator of PTL 1 has a disadvantage in that it needs a dedicated member, i.e. a pad or net, for preventing adhesion of the adhesive substance layer to the top region of the nose bridge.

Therefore, an object of this invention is to provide a nasal dilator comprising a flexible strip, an adhesive substance layer formed on one surface of the flexible strip and a release liner covering the adhesive substance layer, which is used with the release liner removed from the adhesive substance layer, the adhesive substance layer adhered to the nose bridge and the adhesive substance layer prevented from adhering to a top region of the nose bridge, and which does not need a dedicated member for preventing adhesion of the adhesive substance layer to the top region of the nose bridge.

### Solution to Problem

In accordance with this invention, there is provided a nasal dilator comprising a flexible strip, an adhesive substance layer formed on one surface of the flexible strip and a release liner covering the adhesive substance layer, wherein the adhesive substance layer is formed on entire surface of said one surface of the flexible strip, the release liner is divided into three pieces, a first of which covers one longitudinal end region of the adhesive substance layer, a second of which covers longitudinal middle region of the adhesive substance layer and a third of which covers another longitudinal end region of the adhesive substance layer, and wherein the first piece and the third piece of the release liner are removed from the adhesive substance layer and the second piece of the release liner is left adhering to the longitudinal middle region of the adhesive substance layer at the time of use of the nasal dilator.

### Advantageous Effects of Invention

When the nasal dilator is used with both of the longitudinal end pieces, i.e. the first and the third pieces, of the release liner removed from the adhesive substance layer and the longitudinal middle piece, i.e. the second piece, of the release liner left adhering to the adhesive substance layer, the longitudinal middle piece of the release liner abuts the top region of the nose bridge and the adhesive substance layer does not adhere to the top region of the nose bridge. Therefore, the adhesive substance layer does not peel skin of the top region of the nose bridge and the top region of the nose bridge is not damaged when the nasal dilator is removed from the nose bridge. It suffices to divide the release liner into three pieces and no dedicated member is necessary for preventing adhesion of the adhesive substance layer to the top region of the nose bridge.

### BRIEF DESCRIPTION OF DRAWINGS

FIG. 1 is an exploded perspective view of a nasal dilator in accordance with a preferred embodiment of this invention.

### DESCRIPTION OF EMBODIMENTS

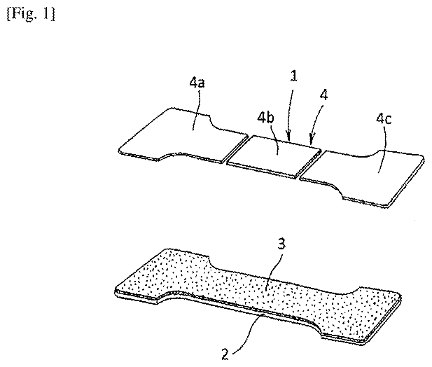

As shown in FIG. 1, a nasal dilator 1 in accordance with a preferred embodiment comprises a flexible strip 2, an adhesive substance layer 3 formed on one surface of the flexible strip 2 and a release liner 4 adhering to and covering the adhesive substance layer 3. The flexible strip 2 is a single or composite material member made of paper, cloth, plastic or the like. The release liner 4 is made of paper, plastic, or the like. The adhesive substance layer 3 is formed on entire surface of said one surface of the flexible strip 2. The release liner 4 is divided into three pieces, namely, a first piece 4a covering one longitudinal end region of the adhesive substance layer 3, a second piece 4b covering longitudinal middle region of the adhesive substance layer 3 and a third piece 4c covering another longitudinal end region of the adhesive substance layer 3.

When the nasal dilator 1 is used, the first piece 4a and the third piece 4c of the release liner 4 are removed from the adhesive substance layer 3, the second piece 4b of the release liner 4, which is left adhering to the longitudinal middle region of the adhesive substance layer 3, is abutted on a top region of the nose bridge, and the two longitudinal end regions of the adhesive substance layer 3 are adhered to opposite side regions of the nose bridge. Restoring force of the flexible strip 2 bent along the nose bridge lifts the opposite side regions of the nose bridge to which the two longitudinal end regions of the adhesive substance layer 3 are adhered. Thus, the nasal cavity is expanded so as to relieve nasal blockage.

When the first piece 4a and the third piece 4c of the release liner 4 are removed from the adhesive substance layer 3 and the second piece 41) of the release liner 4 is left adhering to the longitudinal middle region of the adhesive substance layer 3 during use of the nasal dilator 1, the longitudinal middle portion of the release liner 4, i.e. the second piece 4b of the release liner 4, abuts the top region of the nose bridge so that the adhesive substance layer 3 does not adhere to the top region of the nose bridge. Therefore, the adhesive substance layer 3 does not peel skin of the top region of the nose bridge and the top region of the nose bridge is not damaged when the nasal dilator 1 is removed from the nose bridge.

Opposite longitudinal end regions of the adhesive substance layer 3 adhere to opposite side regions of the nose bridge.

Therefore, the nasal dilator 1 can exhibit good performance, Dividing the release liner 4 into three pieces 4a. 413 and 4c suffices and no dedicated member is necessary for preventing adhesion of the adhesive substance layer 3 to the top region of the nose bridge.

### INDUSTRIAL APPLICABILITY

This invention can be widely used for nasal dilators.

### REFERENCE SIGNS LIST

1 Nasal dilator
2 Flexible strip
3 Adhesive substance layer
4 Release liner
4a First piece
4h Second piece
4e Third piece

1. A nasal dilator comprising a flexible strip, an adhesive substance layer formed on entire of one surface of the flexible strip and a release liner covering the adhesive substance layer, wherein the release liner is divided into a first piece covering one longitudinal end region of the adhesive substance layer and removed from the adhesive substance layer at the time of use of the nasal dilator, a second piece covering longitudinal middle region of the adhesive substance layer and left adhering to the longitudinal middle region of the adhesive substance layer at the time of use of the nasal dilator and a third piece covering another longitudinal end region of the adhesive substance layer and removed from the adhesive substance layer at the time of use of the nasal dilator.
